(19) United States
(12) Patent Application Publication
(10) Pub. No.: US 2011/0236466 A1
(43) Pub. Date: Sep. 29, 2011

(54) CHEMO VECTOR THERAPY TO DELIVER CHEMOTHERAPY MOLECULES TO SPECIFIC CELLS TO MANAGE BREAST CANCER, OTHER CANCERS AND INFLAMMATORY DISORDERS

(76) Inventors: Lane Bernard Scheiber, Annandale, VA (US); Lane Bernard Scheiber, II, Grosse Ile, MI (US)

(21) Appl. No.: 12/730,222

(22) Filed: Mar. 23, 2010

(51) Int. Cl. A61K 9/127; A61K 9/16; A61K 31/4985

(52) U.S. Cl. 424/450; 424/490; 424/491; 514/249

(57) ABSTRACT

The innovative treatment strategy described here utilizes configurable microscopic medical payload delivery devices to act as a transport means to deliver chemotherapy molecules to specific cell types in the body. Utilizing probes present on the exterior of the transport device, transport device locates specific target cell types in the body. Once a specific target cell type has been encountered, the configurable microscopic medical payload delivery device inserts its payload of chemotherapy molecules into the target cell type. By delivering chemotherapy molecules into specific cells, the growth of the specific cells can be stifled. Delivering chemotherapy molecules into specific cancer cells inhibits the rate of growth and rate of cell reproduction of the cancer cells. Utilizing configurable microscopic medical payload delivery devices to insert chemotherapy into targeted cancer cells or inflammatory cells effectively manages cancer, inflammatory arthritis and other inflammatory conditions, while preventing unwanted side effects.

### CROSS-REFERENCE TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2010 Lane B. Scheiber and Lane B. Scheiber II. A portion of the disclosure of this patent document contains material which is subject to copyright protection. The copyright owners have no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device intended to manage breast cancer, other cancers and inflammatory disorders utilizing a configurable microscopic medical payload delivery device to insert one or more chemotherapy molecules into one or more specific cell types.

2. Description of Background Art

Methotrexate has been used for decades to treat various forms of cancer. For over twenty years rheumatologists have treated inflammatory arthritis with methotrexate. Low dose methotrexate is used to suppress the growth and division of synovial cells. The benefits of using methotrexate to treat conditions such as rheumatoid arthritis and psoriatic arthritis was first recognized when patients with rheumatoid arthritis and cancer were treated with high dose methotrexate in an attempt to manage the cancer. In addition to retarding the growth of cancer cells, the methotrexate suppressed the growth of synovial cells that are responsible for the destructive features of rheumatoid arthritis.

Methotrexate is an antimetabolite of the B vitamin. Methotrexate inhibits dihydrofolate reductase (DHFR). Dihydrofolates must be reduced to tetrahydrofolic acid by the enzyme DHFR in the process of deoxyribonucleic acid synthesis. Methotrexate therefore inhibits synthesis of nucleic acids, which in turn inhibits cellular replication. Actively proliferating cells such as malignant cells, bone marrow, fetal cells, buccal mucosa cells, intestinal mucosa cells, and rheumatoid arthritis activated synovial cells are generally more sensitive to the presence of methotrexate. The rate of cellular proliferation in malignant tissues is generally greater than in normal tissues. Methotrexate generally impairs malignant or inflammatory cell growth without irreversibly damaging normal tissues. In many patients, treatment with oral or injectable methotrexate does result in significant unwanted side effects, which prevents effective use of the drug in these patients. Methotrexate is principally excreted from the body by the kidneys.

When utilized to manage an inflammatory arthritis such as rheumatoid arthritis, suppressing folate production suppresses synovial tissue proliferation, which suppresses erosive joint changes. Treatment of rheumatoid arthritis utilizing methotrexate is a strategy of long-term suppression of the disease. Patients with rheumatoid arthritis will often take methotrexate indefinitely.

Methotrexate generally is categorized as a chemotherapy and also an antimetabolite.

Cancer cells generally exhibit a rate of growth and replication that exceeds that of normal healthy cells. Cancer cells whose rate of cell growth and cell division can be slowed down to match that of normal healthy cells would result in such a cancer does not pose a significant health risk to the body.

Breast cancer is a devastating diagnosis for women and men. The survival rate for breast cancer patients has risen significantly over the years as treatment strategies have evolved and improved. Still, the diagnosis or even the threat of breast cancer strikes fear in many women and men around the world. If the growth or rate of cell division of breast cancer cells could be reduced to the same rate as normal cells in breast tissue, then the resultant threat that the occurrence of breast cancer causes would be greatly diminished for the patient, family and friends.

Present medical research is attempting to utilize viruses to deliver genetic information into cells. Research in the field of ‘gene therapy’ has involved certain naturally occurring viruses. Some of the common viral vectors that have been investigated include: Adeno-associated virus, Adenovirus, Alphavirus, Epstein-Barr virus, Gammaretrovirus, Herpes simplex virus, Letivirus, Poliovirus, Rhabdovirus, Vaccinia virus. Naturally occurring virus vectors are limited to the naturally occurring external probes that are affixed to the outer wall of the virus. The external probes fixed to the outside wall of a virus virion dictate which type of cell the virus can engage and infect. Therefore, as an example, the function of the adenovirus, a respiratory virus, is strictly limited to engaging and infecting specific lung cells. Used as a medical treatment device, the adenovirus can only deliver gene therapy to specific lung cells, which severely limits this vector's usefulness as a deliver device. The therapeutic function of all naturally occurring viral vectors is limited to delivering a DNA or RNA payload to the cell type the viral vector naturally targets as its host cell.

Naturally occurring viruses also have the disadvantage of being susceptible to detection and elimination by a body's immune system. Viruses have been infecting humans for hundreds of thousands of years. A human's immune system, comprised of an innate system and an adaptable system, is very complex, and very efficient at detecting the presence of most naturally occurring viruses when such a virus breaches the outer perimeter of the body. The human immune system is quite capable of generating a vigorous response to most intruding viruses, attacking and neutralizing virus virions whenever a virus virion physically exists are outside the exterior wall of the virus's host cell. If gene therapy, in its current state, were to become a clinical therapeutic tool, the naturally occurring viruses selected for gene therapy research will have limited effectiveness. Once a naturally occurring viral vector is introduced into the body, the body's the immune system will most likely detect the intrusion, quickly engage and eliminate the viral vectors, possibly before any one of the vectors is able to deliver its payload to its host cell or target cell.

Cichutek, K., 2001 (U.S. Pat. No. 6,323,031 B1) teaches preparation and use of novel lentiviral SiVagm-derived vectors for gene transfer into selected cell types, specifically into proliferatively active and resting human cells.

Cichutek teaches that it is indeed plausible to re-configure an existing virus and use it as a transport vehicle, though Cichutek's specification and claims are too limited to describe a method that will work for all cell types, if indeed if it will work for any cell type.

Cichutek describes vectors for ‘gene transfer’; in the claims the language that is used is ‘genetic information’. Cichutek's claim 1 of the cited patent states ‘A propagation-incompetent SIVagm vector comprising a viral core and a viral envelope, wherein the viral core comprises a simian immunodeficiency virus (SIVagm) viral core of the African vervet monkey Chlorocebus.’ Cichutek's does not describe in his claims any further details of the intended payload other than the stating ‘SIVagm viral core’ in claim 1; in claims 5 & 6 Cichutek describes only ‘genetic information’. Transfer of ‘genetic information’ dramatically limits the useful application of Cichutek's patent in the treatment of medical diseases.

Cichutek does not claim the use of specific glycogen probes to target specific types of cells. Cichutek's approach is dependent upon the probes naturally present on the viral vectors reported in the patent, which will direct the viral vectors to only those cells the viruses naturally use as their host cell. Cichutek's approach is very restrictive, limited to gene transfer to only cells the viruses use as their natural host cell.

It is questionable that Cichutek's approach as described in the specification and claims is feasible. Cichutek's claim 4, states ‘The SIVagm vector of claim 1, wherein the viral envelope further comprises a single chain antibody (scFv) or a ligand of a cell surface molecule.’ By use of the words ‘a’ and ‘or’ in the claim, the claim is limited in the singular, meaning Cichutek claims a single chain antibody or a singular ligand. Singular type antibodies or ligands can be used for cell-to-cell communication, but to open an access portal into a cell and insert a payload into the cell requires two different types of antibodies or ligands. As an example human immunodeficiency virus requires the use of both the gp120 and gp41 probes to open a portal into a T-Helper cell and inserts its viral genome into the T-Helper cell. The gp120 probe engages the CD4+ cell-surface receptor on the T-cell. Once the gp120 probe has successfully engaged a CD4+ cell-surface receptor on the target T-Helper cell, then the HIV virion's gp41 probe can engage either a CXCR4 or a CCR5 cell-surface receptor on the T-Helper cell in order to open up an access portal for HIV to insert its viral genome into a T-Helper cell. It is well documented in the medical literature that a genetic defect leading to an abnormality in the CXCR4 cell-surface receptor prevents HIV virions from opening an access portal and inserting its genetic payload into such T-Helper cells. This genetic defect in the CXCR4 cell-surface receptors offers the subset of people carrying the genetic defect resistance to HIV infection. This example demonstrates the need for at least two types of glycoprotein probes to be present on the surface of a viral vector in order for a viral vector to be capable of opening an access portal and delivering the payload the vector carries into its host cell or target cell.

A delivery system that offered a defined means of targeting specific types of cells would invoke minimal or no response by the innate immune system or the adaptable immune system when present in the body, and a delivery system that would be capable of inserting into cells a wide variety of chemotherapy molecules would significantly improve the current medical treatment options available to clinicians treating patients. Such a strategy would increase the effectiveness of the chemotherapy and would result in a dramatic reduction in unwanted side effects posed by such chemotherapy.

The solution to arriving at a versatile, workable delivery system that will meet the needs of a number of medical treatments involves three important elements. These elements include:
(1) configurable external probes whereby more than one type of protein structure probe or more than one type of glycoprotein probe is to be used to engage and access specific target cell types in order to successfully deliver a payload into a specific cell type,
(2) an exterior envelope comprised of a protein shell or lipid layer or a lipid bilayer expressing the least number of cell-surface markers, such as the use of a stem cell to act as the host cell to manufacture the delivery devices,
(3) configuring the core or center of the vector to enable it to carry and deliver a wide variety of chemotherapy molecules.

Viruses are obligate parasites. Viruses simply represent a carrier of genetic material and by themselves viruses are unable to replicate or carry out any form of biologic function outside their host cell. A ‘virion’ refers to the physical structure of a single complete virus as it exists outside of the host cell; an older term for ‘viral virion’ was ‘virus particle’. Viruses are generally comprised of one or more nested shells constructed of one or more layers of protein, some with a lipid outer envelope, a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's external probes is to locate and engage a host cell's receptors. The virus's surface probes are designed to detect, make contact with and functionally engage one or more receptors located on the exterior of a cell type that will offer the virus the proper environment in which to construct copies of itself. A host cell provides the virus the proper biologic machinery for the virus to successfully replicate itself. Once the virus's genome is inside the host cell, the viral genome takes command of the cell's production machinery and causes the host cell to generate copies of the virus. As the viral copies exit the host cell, these virions set off in search of other host cells to infect.

Naturally occurring viruses exist in a number of differing shapes. The shape of a virus may be rod or filament like, icosahedral, or complex structures combining filament and polygonal shapes. Viruses generally have their outer wall comprised of a protein coat or an envelope comprised of lipids.

An outer envelope comprised of lipids may be in the form of one or two phospholipid layers. When the outer envelope is comprised of two phospholipid layers this is termed a lipid bilayer. For purposes of this text the term ‘lipid’ includes ‘phospholipid’ molecules. A phospholipid is a composite molecule comprised of a polar or hydrophilic region on one end and a nonpolar or hydrophobic region on the opposite end. A lipid bilayer covering a virus, like the membrane of a cell, is constructed with the hydrophilic region of one of the phospholipid layers pointed toward the exterior of the virion and the hydrophilic region of the second phospholipid layer pointed inward toward the center of the virus virion; with the hydrophobic regions of each of the two lipid layers pointed toward each other. The outer envelope of some forms of virus may be comprised of an outer lipid layer or lipid bilayer affixed to a protein matrix for support, the protein matrix being located closer to the center of the virus virion than the lipid layer or lipid bilayer.

Spherical viruses are generally spherical in shape and may be comprised of an outer envelope and one inner shell or an outer envelope and multiple inner shells. Inner shells are approximately spherical in shape; this is because the proteins comprising the protein matrix shell have an irregular shape to their structure. In the case of a spherical virus with an outer envelope and one inner shell, the inner shell is often referred to as a nucleocapsid shell comprised of numerous capsid proteins attached to each other. In the case of a spherical virus being comprised of an outer envelope and multiple inner shells, the outermost inner viral shells may be referred to as comprised of a quantity of matrix proteins, where the innermost shell is referred to as a nucleocapsid and is comprised of a quantity of capsid proteins. The inner protein shells are nested inside each other. The cavity present created by the innermost shell or nucleocapsid is referred to as the ‘core’ or the ‘center of the virus’. Any payload carried by the virus virion is generally carried in the core or center of the virion.

Viruses carry genetic material in the form of deoxyribonucleic acid (DNA) or ribonucleic acid (RNA) as their payload. DNA or RNA payloads are carried in the cavity of the nucleocapsid referred to as the core. A virus is therefore generally considered to be a DNA virus if its genome is comprised of DNA or the virus is considered a RNA virus if its genome is comprised of RNA that acts as genetic instructions to generate copies of the virus. Viruses may also carry enzymes as part of their payload. An enzyme such as ‘reverse transcriptase’ transforms a RNA viral genome into DNA. Protease enzymes modify the viral genome once it has entered a host cell. An integrase enzyme assists a DNA viral genome with insertion into the host cell's nuclear DNA. The entire genetic payload is carried inside cavity created by the virus's nucleocapsid shell.

The probes attached to the exterior of a virus are constructed to engage specific cell-surface receptors on specific cell types in the body. Only a cell that expresses cell-surface receptors that are capable of being engaged by the probes of a specific virus can act as a host for the virus. Viruses generally use two probes to access a host cell. The first probe makes an initial attachment to the host cell, while the action of the virus's second probe often in conjunction with the action of the first probe cause an access portal to be created in the host cell's exterior plasma membrane. Once an access portal is formed, the virus inserts the contents of its payload into the host cell. Once the virus's genome is inside the cytoplasm of the host cell, any enzymes that accompanied the viral genome into the cell, may begin to modify or assist the virus's genome with infecting and taking control of the host cell's biologic functions.

Probes are attached to the exterior envelope of a virus virion. Probes may be in the form of a protein structure or may be in the form of a glycoprotein molecule. For viruses constructed with a protein matrix as its outer envelope, the probes tend to be protein structures. A portion of the protein structure probe is fixed or anchored in the protein matrix, while a portion of the protein structure probe extends out and away from the protein matrix. The portion of the protein structure probe extending out away from the virus virion is referred to as the ‘exterior domain’, the portion anchored in the protein matrix is the ‘transcending domain’. Some protein probes have a third segment that extends through the envelope and exists inside the virus virion, which is referred to as the ‘interior domain’. The exterior domain of a protein structure probe is intended to engage a specific cell-surface receptor on a biologically active cell the virus is targeting as its host cell.

Viruses that utilize a lipid layer as the outer envelope, are constructed with probes that tend to be glycoproteins. A glycoprotein is comprised of a protein segment and a carbohydrate segment. The carbohydrate segment of the glycoprotein molecule is fixed or anchored in the lipid layer of the outer envelope, while the protein segment extends outward and away from the outer envelope. The protein portion of a glycoprotein probe that extends outward and away from the outer envelope of a virus virion is intended to engage a cell-surface receptor on a biologically active cell the virus is targeting as its host cell.

Some forms of viruses that utilize a lipid layer as its envelope use protein structure probes. In this case, the portion of the protein structure probe that extends outward and away from the outer envelope is the ‘exterior domain’, the portion that is anchored in the lipid layer is the ‘transcending domain’ and again some protein structure probes have an ‘interior domain’ that exist inside the virion, which may also help anchor the protein structure probe to the virion. The exterior domain of a protein structure probe that extends outward and away from the outer envelope of a virus virion is intended to engage a cell-surface receptor on a biologically active cell the virus is targeting as its host cell.

When a virus carries a DNA payload and the viral DNA is inserted into the host cell, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate use of the viral RNA by having the RNA converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the nuclear DNA and simply utilize portions of the viral genome to act as messenger RNA. RNA viruses that bypass the host cell's DNA, cause the cell in general to generate copies of the necessary parts of the virus directly from the virus's RNA genome.

The human immunodeficiency virus (HIV) is a RNA virus and has an outer envelope comprised of a lipid bilayer. The lipid bilayer covers a protein matrix consisting of p17^{gag} proteins. Inside the p17^{gag} protein is nested a nucleocapsid comprised of p24^{gag} proteins. Inside the nucleocapsid HIV carries its payload. HIV's genetic payload consists of two single strands of RNA and several enzymes. The enzymes that accompany HIV's genome include ‘reverse transcriptase’, ‘integrase’ and ‘protease’ molecules.

The T-Helper cell acts as HIV's host cell. The HIV virion utilizes two types of glycoprotein probes affixed to its exterior envelope to locate and engage a T-Helper cell. HIV utilizes a glycoprotein probe 120 to locate a CD4 cell-surface receptor on a T-Helper cell. Once an HIV glycoprotein 120 probe has successfully engaged a CD4 cell surface-receptor on a T-Helper cell a conformational change occurs in the glycoprotein 120 probe and a glycoprotein 41 probe is exposed. The glycoprotein 41 probe's intent is to engage a CXCR4 or CCR5 cell-surface receptor on the same T-Helper cell. Once a glycoprotein 41 probe on the HIV virion successfully engages a CXCR4 or CCR5 cell-surface receptor, the HIV virion opens an access portal through the T-Helper cell's outer membrane.

Once the HIV virion has opened an access portal through the T-Helper cell's outer plasma membrane, the HIV virion inserts two positive strand RNA molecules and the associated enzymes it carries into the T-Helper cell. Each RNA strand is approximately 9500 nucleotides in length. Inserted along with the RNA strands are the enzymes reverse transcriptase, protease and integrase. Once the virus's genome gains access to the interior of the T-Helper cell, in the cytoplasm the pair of RNA molecules are transformed to deoxyribonucleic acid by the reverse transcriptase enzyme. Following modification of the virus's genome to DNA, the virus's genetic information migrates to the host cell's nucleus. In the nucleus, with the assistance of the integrase protein, the HIV's DNA becomes inserted into the T-Helper cell's native nuclear DNA. When the timing is appropriate, the now integrated viral DNA is decoded by the host cell's polymerase molecules and the virus's genetic information commands certain cell functions to carry out the replication process to construct copies of the human immunodeficiency virus.

The outer layer of the HIV virion is comprised of a portion of the T-Helper cell's outer cell membrane. In the final stage of the replication process, as a copy of the HIV virion, carrying the HIV genome, buds through the host cell's cell membrane the outer capsid acquires as its exterior envelope, a wrapping of lipid bilayer from the host cell's cell membrane. In the case of HIV, since the surface of the pathogen is covered by an envelope comprised of lipid bilayer taken from the host T-Helper cells, this feature allows the HIV virion the capacity to eluded the two immune systems, since the detectors comprising the innate immune system and the adaptable immune system may find it difficult to distinguish between the surface of an infectious HIV virion and the surface characteristics of a noninfected T-Helper cell.

The Hepatitis C virus (HCV) is a positive sense RNA virus, meaning a type of RNA that is capable of bypassing the need for involving the host cell's nucleus by having its RNA genome function as messenger RNA. Hepatitis C infects liver cells. The Hepatitis C viral genome becomes divided once it gains access to the interior of a liver host cell. Portions of the subdivisions of the Hepatitis C genome directly interact with ribosomes to produce proteins necessary to construct copies of the virus.

HCV belongs to the Flaviviridae family and is the only member of the Hepacivirus genus. There are considered to be at least 100 different strains of Hepatitis C virus based on genome sequencing variability.

HCV is comprised of an outer lipoprotein envelope and an internal nucleocapsid. The genetic payload is carried within the nucleocapsid. In its natural state, present on the surface of the outer envelope of the Hepatitis C virus are probes that detect receptors present on the surface of liver cells. The glycoprotein E1 probe and the glycoprotein E2 probe have been identified to be affixed to the surface of HCV. The E2 probe binds with high affinity to the large external loop of a CD81 cell-surface receptor. CD81 is found on the surface of many cell types including liver cells. Once the E2 probe has engaged the CD81 cell-surface receptor, cofactors on the surface of HCV's exterior envelope engage either or both the low density lipoprotein receptor (LDLR) or the scavenger receptor class B type I (SR-BI) present on the liver cell in order to effect the mechanism to facilitate HCV breaching the cell membrane and inserting its RNA genome payload through the plasma cell membrane of the liver cell into the liver cell. Upon successful engagement of the HCV surface probes with a liver cell's cell-surface receptors, HCV inserts the single strand of RNA and other payload elements it carries into the liver cell targeted to be a host cell. The HCV RNA genome then interacts with enzymes and ribosomes inside the liver cell in a translational process to produce the proteins required to construct copies of the protein components of HCV. The HCV genome undergoes a method of transcription to replicate copies of the virus's RNA genome. Inside the host, pieces of the HCV virus are assembled together and ultimately loaded with a copy of the HCV genome. Replicas of the original HCV then escape the host cell and migrate the environment in search of additional host liver cells to infect and continue the replication process.

The HCV's naturally occurring genetic payload consists of a single molecule of linear positive sense, single stranded RNA approximately 9600 nucleotides in length. By means of a translational process a polyprotein of approximately 3000 amino acids is generated. This polyprotein is cleaved post translation by host and viral proteases into individual viral proteins which include: the structural proteins of C, E1, E2, the nonstructural proteins NS1, NS2, NS3, NS4A, NS4B, NS5A, NS5B, p7 and ARFP/F protein. Hepatitis C virus's proteins direct the host liver cell to construction copies of the Hepatitis C virus. A membrane associated replicase complex consisting of the virus's nonstructural proteins NS3 and NS5B facilitate the replication of the viral genome. The membrane of the endoplasmic reticulum appears to be the site of protein maturation and viral assembly. Once copies of the Hepatitis C Virus are generated, they exit the host cell and each copy of HCV migrates in search of another appropriate liver cell that will act as a host to continue the replication process.

Hepatitis C virus life-cycle demonstrates that copies of a virus virion can be generated by inserting RNA into a host cell that functions as messenger RNA in the host cell. The Hepatitis C viral RNA genome functions as messenger RNA, acting as the template in conjunction with the biologic machinery of a host cell to produce the components that comprise copies of the Hepatitis C virion and the Hepatitis C viral RNA provides the biologic instructions to assemble the components into complete copies of the Hepatitis C virions. The Hepatitis C virus life-cycle clearly demonstrates that viral virions can be manufactured by a host cell without involving the nucleus of the cell.

Deciphering the existence, replication and behavior of viruses provides clear examples of several fundamental concepts, which include: (1) Viruses target specific cells in the body by means of identifying and engaging such target cells utilizing the probes projecting outward from the virus's exterior shell to make contact with cell-surface receptors located on the surface of their host cells, and (2) Viruses are capable of carrying a variety of different types of payloads including DNA, RNA and a variety of proteins.

Current gene therapy approach to attempting to deliver a payload to cells in the body use modified forms of existing viruses to act as transport devices to deliver genetic information. This approach is severely limited by restricting the virus virion to the target only cells the viral vector naturally seeks out and infects. Current gene therapy approach is further limited by using the pre-existing size of naturally occurring viruses, rather than being able to modify the size of the structure to be able to tailor the volumetric carrying capacity of the payload portion of the modified virus. Further gene therapy is restricted to utilizing naturally occurring viruses to deliver only genetic information; it has not previously been appreciated by those skilled in the art that virus-like transport devices might deliver to a variety of specific cell types a wide variety of differing payloads including various chemotherapy drug molecules.

A dramatic necessity, not previously recognized by those expert in the art, is the need to develop a transport vehicle that can be fashioned to seek out specific types of cells and deliver to these cells chemotherapy molecules to treat cancer and inflammatory disorders by inhibiting the rate cell growth and replication of these specific target cells. The exterior envelope of a transport vehicle should be constructed so as not to alert either component of the immune system of its presence to prevent rejection of these transport vehicles. Transport vehicles should be capable of being configured to target any specific cell type and engage and deliver their chemotherapy payload only to that specific cell type. To this point, no such device or process has been documented in the literature.

### BRIEF SUMMARY OF THE INVENTION

Utilization of configurable microscopic medical payload delivery devices to deliver chemotherapy molecules to specific cell types facilitates a dramatic new approach to managing cancer and inflammatory disorders. By selecting the type of probes that will effectively engage cell-surface receptors on target cells and fixing these probes on the surface of the configurable microscopic medical payload delivery devices, specific types of cells can be targeted. By utilizing configurable microscopic medical payload delivery devices to deliver chemotherapy molecules to specific cell types, the rate of cell growth and rate of cell replication can be inhibited without provoking unwanted side effects in other cells. A wide variety of cancers and inflammatory disorders are treatable by utilizing this new and unique approach.

### DETAILED DESCRIPTION

Future medical treatment includes the aggressive, widespread utilization of configurable microscopic medical payload delivery devices (CMMPDD) to deliver chemotherapy molecules directly to specific targeted cell types in the body.

The configurable microscopic medical payload delivery device transporting chemotherapy molecules represents a very versatile medical treatment delivery device. CMMPDD is used to deliver chemotherapy molecules to a wide variety of cancer cells and inflammatory cells. Utilizing CMMPDD to deliver chemotherapy molecules to cancer cells represents a new means to manage cancer. Using CMMPDD to deliver chemotherapy molecules to inflammatory arthritis represents a new means to treat inflammatory arthritis. By delivering chemotherapy molecules directly to targeted cells and only to the targeted cells by virus-like transport devices, the configurable microscopic medical payload delivery devices represent a significant advancement over current chemotherapy treatment techniques in that this strategy avoids many of the unwanted side effects that conventional chemotherapy cause.

For purposes of this text an ‘external envelope’ refers to the outermost covering of a virus or a virus-like transport device or a configurable microscopic medical payload delivery device. The external envelope may be comprised of a lipid layer, a lipid bilayer, the combination of a lipid layer affixed to a protein matrix or the combination of a lipid bilayer affixed to a protein matrix. A protein matrix is equivalent to a protein shell and may be referred to as a protein matrix shell. The terms protein matrix, protein shell, protein matrix shell are equivalent to the term capsid, where the term capsid is meant to represent ‘a protein coat or shell of a virus particle, surrounding the nucleic acid or nucleoprotein core’. The term ‘particle’ is equivalent to the term ‘virion’.

For purposes of this text an ‘internal shell’ refers to a protein matrix shell nested inside the external envelope. The innermost protein matrix shell is termed the nucleocapsid. The proteins that comprise the nucleocapsid are termed capsid proteins. In the cavity created by the nucleocapsid, referred to as the center or core of the nucleocapsid, is where the payload of chemotherapy molecules is carried.

For purposes of this text ‘external probes’ are molecular structures that are utilized to locate and engage cell-surface receptors on biologically active cells. External probes are generally comprised of a portion which is anchored or fixed in the external envelope and a second portion that extends out and away from the external envelope. The portion of the external probe that extends out and away from the external envelope is intended to make contact and engage a cell-surface receptor located on a biologically active cell. External probes may be comprised solely of a protein structure or an external probe may be a glycoprotein molecule.

For purposes of this text ‘glycoprotein molecule’ refers to a molecule comprised of a carbohydrate region and a protein region. Glycoprotein molecules that act as probes are generally anchored or fixed to a lipid layer utilizing the carbohydrate portion of the molecule as an anchor. The protein portion of the glycoprotein molecule which extends outward and away from the exterior envelope the glycoprotein has been affixed such that the protein region may function as a probe to locate and attach to the cell-surface receptor it was created to engage.

The concept of configurable microscopic medical payload delivery devices is modeled after naturally existing viruses. Configurable microscopic medical payload delivery devices in general are spherical in shape; though other shapes may be used as function might warrant the use of a particular shape. The spherical configurable microscopic medical payload delivery devices are comprised of an exterior envelope and one or more inner nested protein shells. A quantity of exterior protein structure probes and/or glycoprotein probes are anchored in the exterior lipid envelope and a portion extends out and away from the exterior lipid envelope. Nesting of protein shells refers to progressively smaller diameter shells fitting snugly inside protein shells of a larger diameter. Inside the inner most protein shell, referred to as the nucleocapsid, is a cavity referred to as the core of the device. The core of the device is the space where the medically therapeutic payload the device carries is located. The payload of the device is comprised of chemotherapy molecules.

Configurable microscopic medical payload delivery devices (CMMPDD) target specific types of cells in the body. Configurable microscopic medical payload delivery devices engage specific types of cells by the configuration of probes affixed to the exterior envelope of the CMMPDD. By fixing specific probes to the exterior envelope of the CMMPDD, these probes intended to engage and attach only to specific cell-surface receptors located on certain cell types in the body, the CMMPDD will deliver its payload to only those cell types that express compatible and engagable specific cell-surface receptors. In a similar fashion where the exterior probes of a naturally occurring virus engage specific cell-surface receptors present on the surface of the virus's host cell and only the designated host cell, the CMMPDD's exterior probes are configured to engage cell-surface receptors on a specific type of target cell and only those cells. In this manner, the payload of chemotherapy drug molecules carried by CMMPDD will be delivered only to specific types of cells in the body. The configuration of the exterior probes on the surface of a CMMPDD will vary as needed so as to effect the CMMPDD delivery of chemotherapy drug payloads to the specific cell types as needed to effect a particular predetermined medical treatment.

The size of configurable microscopic medical payload delivery devices is dependent upon the diameter of the inner protein matrix shells and this is dictated by the volume size of the payload the CMMPDD is required to carry and deliver to a target cell. The diameter of each inner protein matrix shell is governed by the number of protein molecules utilized to construct the protein matrix shell at the time the protein matrix shell is generated. Increasing the number of proteins that comprise a protein matrix shell, increases the diameter of the protein matrix shell. When applicable an external lipid envelope wraps around and covers the outermost protein matrix shell. The larger the volume of the core of the CMMPDD, the greater the physical size payload the CMMPDD is able to carry. The size of the configurable microscopic medical payload delivery device is to be the size of cell (approximately 10⁻⁴ m in diameter) or less, generally detectable by a light microscope or, as needed, an electron microscope. The size of the CMMPDD is not to be too large such that it would generate a burden to the body by damaging organ tissues through clogging blood vessels or glomeruli in the kidneys. The dimensions of each type of CMMPDD are to be tailored to the mission of the CMMPDD, which takes into account factors such as the type of target cell, the size of the payload that is to be delivered to the target cells and the length of time the CMMPDD may have to engage the target cell.

Being enveloped in an external lipid layer, configurable microscopic medical payload delivery devices possess the advantage of having their exterior appear similar to the plasma membrane that acts as an outside covering for the cells that comprise the body. By appearing similar to existing plasma membranes, the CMMPDDs appear similar to naturally occurring structures found in the body. CMMPDD's are afforded the capability to avoid detection by a body's immune system because the exterior of the CMMPDD mimics the cells comprising the body and the surveillance elements of the immune system find it difficult to discern between the CMMPDD and naturally occurring cells comprising the body.

To carry out the process of manufacturing a configurable microscopic medical payload delivery device, a primitive cell such as a stem cell is selected. The reason for utilizing primitive cells such as stems cells as the host cell, is that the CMMPDD acquires its outer envelope from the host cell and the more primitive the host cell, the fewer in number the identifying protein markers are present on the surface of the CMMPDD. The fewer the identifying surface proteins present on the outer envelope of the CMMPDD, the less likely a body's immune system will identify the CMMPDD as an intruder and therefore less likely the body's immune system will react to the presence of the CMMPDD and reject the CMMPDD by attacking and neutralizing the CMMPDD.

Stem cells used as host cells to manufacture quantities of CMMPDD product are selected per histocompatibility markers present on their surface. Certain histocompatibility markers present on the surface of the final CMMPDD product will be less likely to cause a reaction in a specific patient based on the genetic profile of the patient's histocompatibility markers. A similar histocompatibility match is done when donor organs are selected to be given to recipients to avoid rejection of the donor organ by the recipient's immune system.

The selected stem cells used to manufacture configurable microscopic medical payload delivery devices goes through several steps of maturation before it is capable of generating therapeutic CMMPDD product. Messenger RNA would be inserted into the host stem cell that would code for the general physical outer structures of the CMMPDD. Messenger RNA would be inserted into the host that would generate surface probes that would target the cell-surface receptors on specific target cell types. Messenger RNA would be inserted into the host that would be used to generate the payload of chemotherapy molecules or the chemotherapy molecules are introduced into the CMMPDD post production of the CMMPDD based on the size and complexity of the chemotherapy molecules. Similar to how copies of a naturally occurring virus, such as the Hepatitis C virus or HIV, are produced, assembled and released from a host cell, copies of the CMMPDD would be produced, assembled and released from a host cell. Once released from the stem cell functioning as a de facto host cell, the copies of the CMMPDD would be collected, then pooled together to produce a therapeutic dose that results in a medically beneficial effect.

The stem cells used as host cells are suspended in a broth of nutrients and are kept at an optimum temperature to govern the rate of production of the CMMPDD product. Similar to the natural production of the Hepatitis C virus, the configurable microscopic medical payload delivery devices ‘production genome’ is introduced into the host stem cells. The configurable microscopic medical payload delivery devices production genome carries genetic instructions to cause the host cells to manufacture the configurable microscopic medical payload delivery devices' outer protein wall, the inner protein matrixes, the surface probes the configurable microscopic medical payload delivery device is to have affixed to its outer envelope, the chemotherapy molecules the configurable microscopic medical payload delivery devices are to carry, and the instructions to assemble the various pieces into the final form of the configurable microscopic medical payload delivery devices along with the instructions to activate the budding process. The resultant configurable microscopic medical payload delivery devices are collected from the nutrient broth surrounding the host cells and placed together into doses to be used as a treatment for a medical disease.

The ‘production genome’ are an array of messenger RNAs that are directly translated by the host cell's ribosomes. The production genome dictates the characteristics of the final version of the CMMPDD that buds from the host stem cell and is released and is to be utilized as a medical treatment. The production genome is specifically tailored to code for the surface probes that will seek and engage a specific type of target cell. The production genome also carries the instructions to code for the production of the type of chemotherapy molecules to be delivered to the specific type of target cell. The ‘production genome’ varies depending upon the configuration of the CMMPDD and the type of chemotherapy molecules the CMMPDD will transport to effect a specific medical treatment in a specific type of cell.

The configurable microscopic medical payload delivery device transporting chemotherapy molecules represents a very versatile medical treatment delivery device. CMMPDD is used to deliver chemotherapy molecules to a wide variety of cancer cells and inflammatory cells. Utilizing CMMPDD to deliver chemotherapy molecules to cancer cells represents a new means to manage cancer. Using CMMPDD to deliver chemotherapy molecules to inflammatory arthritis represents a new means to treat inflammatory arthritis.

As an example of this method, to treat breast cancer utilizing configurable microscopic medical payload delivery devices to deliver to breast cells chemotherapy such as the antimetabolite methotrexate, the following production process is followed in the lab: (1) human stem cells are selected. (2) Into the selected stem cells is placed the production genome constructed, in this case, specifically as a means to treat breast cancer. The RNA production genome contains genetic instructions to cause the host stem cells to manufacture the configurable microscopic medical payload delivery devices' outer protein wall, the inner protein matrix, surface probes to include glycoprotein probes that engage estrogen cell-surface receptors present on the surface of breast cells, and the payload of chemotherapy molecules; and the biologic instructions to assemble the components into the final form of the configurable microscopic medical payload delivery devices; and the biologic instructions to activate the budding process. (3) Upon insertion of the RNA production genome dedicated to producing a configurable microscopic medical payload delivery devices to transport chemotherapy molecules, into the host stem cells, host stem cells respond by (i) simultaneously translating the different segments of the RNA production genome to produce the proteins that comprise the exterior protein wall, the inner protein matrix shell molecules, the surface probes to seek out and engage breast cells, the chemotherapy molecules, and (ii) decoding the RNA instructions to assemble the components into the configurable microscopic medical payload delivery devices. (4) Upon assembly, the configurable microscopic medical payload delivery devices bud through the cell membrane of the host stem cell. (5) At the time of the budding process, the configurable microscopic medical payload delivery devices acquire an outside envelope wrapped over the outer protein shell, this outer envelope comprised of a portion of the plasma membrane from the host stem cell as the configurable microscopic medical payload delivery devices exit the host cell. (6) In some cases dependent upon the treatment application and how tightly packed the configurable microscopic medical payload delivery devices need to be packed with chemotherapy molecules, such chemotherapy molecules may be present in the nutrient broth and diffuse into the configurable microscopic medical payload delivery devices. (7) The resultant configurable microscopic medical payload delivery devices are collected from the nutrient broth surrounding the host stem cells. (8) The configurable microscopic medical payload delivery devices are washed in sterile solvent to remove contaminants. (9) The configurable microscopic medical payload delivery devices are removed from the sterile solvent and suspended in a hypoallergenic liquid medium. (10) The configurable microscopic medical payload delivery devices are separated into individual quantities to facilitate storage and delivery to physicians and patients. (11) The configurable microscopic medical payload delivery devices transported in the hypoallergenic liquid medium is administered to a patient with a diagnosis of breast cancer per injection in a dose that is tailored to receiving patient's severity of breast cancer. (12) Upon being injected into the body, the configurable microscopic medical payload delivery devices migrate to the breast tissues by means of the patient's blood stream. (13) Upon the configurable microscopic medical payload delivery devices reaching the breast cells, the configurable microscopic medical payload delivery devices engage the cell-surface receptors located on the breast cells and insert the payload they carry into the breast cells. The payload, in this case being the chemotherapy methotrexate. Methotrexate molecules inhibit dihydrofolate reductase and therefore prevents breast cells synthesizing nucleic acids, therefore prevents cell division, which prevents the cancer cells from replicating but does not interfere with normal breast cells.

In a similar fashion, configurable microscopic medical payload delivery devices can be fashioned to deliver a payload of a specific chemotherapy molecule to any type of cancer cell or inflammatory cell in the body. Different cell types express different cell-surface markers on the exterior of their plasma membrane. The differing configurations of cell-surface markers on differing types of cells distinguish one cell type from another cell type. By configuring the exterior probes that extend from the surface of the configurable microscopic medical payload delivery device to seek out and engage specific cell-surface receptors present on normal cells and cancer cells payloads of chemotherapy can be delivered to specific cells in the body.

Rheumatoid arthritis is an inflammatory disease affecting millions of people worldwide. The chemotherapy drug methotrexate has been used for over twenty years, and is used quite extensively as a treatment of rheumatoid arthritis. Oral and injectable forms of the methotrexate are used to suppress the growth and cell division of synovial tissues. Oral and injectable methotrexate causes many unwanted side effects. Utilizing configurable microscopic medical payload delivery devices to deliver methotrexate molecules specifically to synovial cells and only to synovial cells, increases the efficacy of methotrexate and reduces or eliminates the unwanted side effects of methotrexate.

### CONCLUSIONS, RAMIFICATION, AND SCOPE

Accordingly, the reader will see that the configurable microscopic medical payload delivery device to deliver chemotherapy molecules to specific targeted cell types provides advantages over existing art by: (1) being a delivery device that seeks out specific types of cells, (2) by being a delivery device that is versatile enough to deliver a variety of potential chemotherapy molecules to accomplish various medical treatments, (3) by being a device that delivers chemotherapy molecules only to targeted cells, thus avoiding unwanted side effects that current chemotherapy causes, and (4) by being a delivery device constructed with a surface envelope that will avoid detection by the innate immune system and the adaptable immune system so as not to activate either immune system to its presence; for these reasons this represents a new and unique medical delivery device that has never before been recognized nor appreciated by those skilled in the art.

Although the description above contains specificities, these should not be construed as limiting the scope of the invention but as merely providing illustrations of some of the presently preferred embodiments of the invention.

Thus the scope of the invention should be determined by the appended claims and their legal equivalents, rather than by the examples given.

### NUMBER OF DRAWINGS

0

The terms and expressions which are employed here are used as terms of description and are not of limitation and there is no intention, in the use of terms and expressions, of excluding equivalents of the features presented, and described, or portions thereof, it being recognized that various modifications are possible in the scope of the invention or process as claimed.

What is claimed:

1. A configurable microscopic medical payload delivery device comprised of:
(a) an exterior envelope,
(b) a quantity of interior shells,
(c) a quantity of configurable exterior probes attached in a manner that a segment of said exterior probes is to project out and away from said exterior envelope, while a segment of said exterior probes is embedded in said exterior envelope, and
(d) a medically therapeutic payload comprised of a quantity of chemotherapy molecules,
whereby said configurable microscopic medical payload delivery device is intended to deliver said quantity of chemotherapy molecules to a specific cell type in order to produce a medically beneficial effect,
whereby said exterior probes are intended to engage specific cell-surface receptors on said specific cell type,
whereby said chemotherapy molecules are carried as said medically therapeutic payload in the core of said configurable microscopic medical payload delivery device,
whereby said interior shells are versatile enough in their construction to transport within the core a wide variety of types of said chemotherapy molecules as said medically therapeutic payload to said specific cell types,
whereby the utilization of said configurable microscopic medical payload delivery device to deliver said chemotherapy molecules directly to said specific cell types significantly increases the effectiveness of said chemotherapy molecules and significantly reduces the unwanted side effects compared to standard chemotherapy treatment strategies.

2. The configurable microscopic medical payload delivery device in claim 1 wherein said external envelope is selected from the group consisting of a lipid layer, a lipid bilayer, a protein matrix, a lipid layer affixed to a protein matrix, and a lipid bilayer affixed to a protein matrix.

3. The configurable microscopic medical payload delivery device in claim 1 wherein said external envelope is comprised of a quantity of lipid layers and a quantity of protein matrix shells.

4. The configurable microscopic medical payload delivery device in claim 1 wherein said quantity of internal shells are comprised of a quantity of nested sphere-like concentric protein matrix shells.

5. The quantity of lipid layers in claim 4 wherein said quantity of lipid layers is a quantity of phospholipid layers.

6. The configurable microscopic medical payload delivery device in claim 1 wherein said exterior probes are comprised of a quantity of protein molecules and a quantity of glycoprotein molecules.

7. The protein molecules in claim 6 wherein said protein molecules are comprised of a segment of said protein molecules which extends outward and away from said exterior envelope, attached to a segment of said protein molecule which is embedded in said exterior envelope to hold said protein molecule affixed to said exterior envelope,
whereby said segment of said protein molecules which extends outward and away from said exterior envelope is intended to engage said specific cell-surface receptors on said specific cell type.

8. The protein molecules in claim 6 wherein said protein molecules are comprised of a plurality of protein molecules,
whereby, at least two differing configurations of said protein molecules may be needed to successfully engage said specific cell type with one type of configuration of said protein molecule engaging one type of said specific cell-surface receptor, while a differing type of configuration of said protein molecule is required to engage a differing type of said specific cell-surface receptor in order for said configurable microscopic medical payload delivery device to insert said quantity of chemotherapy molecules said configurable microscopic medical payload delivery device carries into intended said specific cell type.

9. The glycoprotein molecules in claim 6 wherein said glycoprotein molecules are comprised of a protein segment which extends outward and away from said exterior envelope, which is attached to a carbohydrate segment, said carbohydrate segment being embedded in said exterior envelope to hold said glycoprotein molecule affixed to said exterior envelope,
whereby said protein segment which extends outward and away from said exterior envelope is intended to engage said specific cell-surface receptor on said specific cell type.

10. The glycoprotein molecules in claim 6 wherein said glycoprotein molecules are comprised of a plurality of glycoprotein molecules,
whereby, at least two differing configurations of said glycoprotein molecules may be needed to successfully engage said specific cell type with one type of configuration of said glycoprotein molecule engaging one type of said specific cell-surface receptor, while a differing type of configuration of said glycoprotein molecule is required to engage a differing type of said specific cell-surface receptor in order for said configurable microscopic medical payload delivery device to insert said quantity of chemotherapy molecules into said specific cell type.

11. The chemotherapy molecules in claim 1 wherein said chemotherapy molecules are molecules of methotrexate,
whereby said methotrexate molecules are carried as said medically therapeutic payload in the core of said configurable microscopic medical payload delivery device.

12. A method for attenuating the rate of growth of specific types of cells comprising:
(a) providing a quantity of protein shells,
(b) covering said protein shells with an exterior envelope,
(c) fixing a quantity of exterior probes to said exterior envelope, and
(d) positioning a quantity of chemotherapy molecules inside the cavity created by the inner most said protein shell,
whereby said quantity of protein shells covered with said exterior envelope with said quantity of exterior probes affixed to said exterior envelope, with said chemotherapy molecules carried in said cavity created by said inner most protein shell, engages said specific types of cells and inserts said chemotherapy molecules into said specific types of cells,
whereby said specific types of cells are located by said exterior probes engaging specific cell-surface receptors on said specific types of cells,
whereby inserting said chemotherapy molecules into said specific types of cells will attenuate the growth rate of said specific types of cells,
whereby the utilization of said method which delivers said chemotherapy molecules directly to said specific types of cells significantly increases the effectiveness of said chemotherapy molecules and significantly reduces the number of unwanted side effects compared to standard chemotherapy treatment strategies.

13. The method for attenuating the rate of growth of specific types of cells in claim 12 wherein said external envelope is selected from the group consisting of a lipid layer, a lipid bilayer, a protein matrix, a lipid layer affixed to a protein matrix, and a lipid bilayer affixed to a protein matrix.

14. The configurable microscopic medical payload delivery device in claim 12 wherein said external envelope is comprised of a quantity of lipid layers and a quantity of protein matrix shells.

15. The quantity of lipid layers in claim 14 wherein said quantity of lipid layers is a quantity of phospholipid layers.

16. The method for attenuating the rate of growth of specific types of cells in claim 12 wherein said quantity of protein shells is comprised of a quantity of nested sphere-like concentric protein matrix shells.

17. The method for attenuating the rate of growth of specific types of cells in claim 12 wherein said exterior probes are comprised of a quantity of protein molecules and a quantity of glycoprotein molecules.

18. The protein molecules in claim 17 wherein said protein molecules are comprised of a segment of said protein molecule which extends outward and away from said exterior envelope, which is attached to a segment of said protein molecule embedded in said exterior envelope to hold said protein molecule affixed to said exterior envelope,
whereby said segment of said protein molecule which extends outward and away from said exterior envelope is intended to engage said specific cell-surface receptor on said predetermined biologically active cells of a particular type.

19. The protein molecules in claim 17 wherein said protein molecules are comprised of a plurality of protein molecules,
whereby, at least two differing configurations of said protein molecules may be needed to successfully engage said predetermined biologically active cells of a particular type with one type of configuration of said protein molecule engaging a type of said specific cell-surface receptor, while a differing type of configuration of said protein molecule is required to engage a differing type of said specific cell-surface receptor in order for said configurable medical treatment payload delivery means to insert said quantity of chemotherapy molecules into intended said predetermined biologically active cells of a particular type.

20. The glycoprotein molecules in claim 17 wherein said glycoprotein molecules are comprised of a protein segment which extends outward and away from said exterior envelope, which is attached to a carbohydrate segment, said carbohydrate segment intended to be embedded in said exterior envelope to hold said glycoprotein molecule affixed to said exterior envelope,
whereby said protein segment which extends outward and away from said exterior envelope is intended to engage said specific cell-surface receptor on said predetermined biologically active cells of a particular type.

21. The glycoprotein molecules in claim 17 wherein said glycoprotein molecules are comprised of a plurality of glycoprotein molecules,
whereby, at least two differing configurations of said glycoprotein molecules may be needed to successfully engage said predetermined biologically active cells of a particular type with one type of configuration of said glycoprotein molecule engaging a type of said specific cell-surface receptor, while a differing type of configuration of said glycoprotein molecule is required to engage a differing type of said specific cell-surface receptor in order for said configurable medical treatment payload delivery means to insert said quantity of chemotherapy molecules into said predetermined biologically active cells of a particular type.

22. The chemotherapy molecules in claim 12 wherein said chemotherapy molecules are molecules of methotrexate,
whereby said methotrexate molecules are carried as said medically therapeutic payload in the core of said configurable microscopic medical payload delivery means.
